(19) United States
(12) Patent Application Publication
(10) Pub. No.: US 2023/0000865 A1
(43) Pub. Date: Jan. 5, 2023

(54) PHARMACEUTICAL COMPOSITION FOR THE TREATMENT OF PULMONARY VASCULAR DISEASE AND/OR CARDIAC DYSFUNCTION IN FONTAN-PALLIATED PATIENTS

(71) Applicant: ACTELION PHARMACEUTICALS LTD, Allschwil (CH)

(72) Inventors: Dénes CSONKA, Allschwil (CH); Wassim FARES, Raritan, NJ (US); Hans HOOGKAMER, Allschwil (CH); Koen TORFS, Beerse (BE)

(21) Appl. No.: 17/779,787

(22) PCT Filed: Nov. 25, 2020
(86) PCT No.: PCT/EP2020/083289

§ 371 Date: May 25, 2022

(30) Foreign Application Priority Data
Nov. 26, 2019 (EP) PCT/EP2019/082586

(51) Int. Cl. A61K 31/506; A61K 31/343; A61K 9/20; A61P 9/00; A61K 31/495

(52) U.S. Cl. CPC A61K 31/506; A61K 31/343; A61K 9/20; A61P 9/00; A61K 31/495

(57) ABSTRACT

The present invention relates to high doses of macitentan (INN), i.e. propylsulfamic acid [5-(4-bromo-phenyl)-6-[2-(5-bromo-pyrimidin-2-yloxy)-ethoxy]-pyrimidin-4-yl]-amide or pharmaceutically acceptable salts, solvates, hydrates or morphological forms thereof for use in the treatment of pulmonary vascular disease and/or cardiac dysfunction in functional single ventricular heart disease patients, especially in Fontan-palliated patients. Moreover, the present invention relates to the use of high doses of macitentan for the manufacture of a medicament as well as to a method for the treatment of said diseases. Further, the present invention relates to a dosage regimen as well as to a combination of macitentan with one or more phosphodiesterase type 5 (PDE5) inhibitors, prostacyclin analogues, prostacyclin receptor agonists or soluble guanylate cyclase stimulators. Besides, the present invention relates to a pharmaceutical composition for the treatment of pulmonary vascular disease and/or cardiac dysfunction in functional single ventricular heart disease patients, especially in Fontan-palliated patients comprising a high dose of macitentan. Moreover, the present invention relates to the use of high doses aprocitentan for the same purpose.

### FIELD OF THE INVENTION

The present invention relates to high doses of macitentan (INN), i.e. propylsulfamic acid [5-(4-bromo-phenyl)-6-[2-(5-bromo-pyrimidin-2-yloxy)-ethoxy]-pyrimidin-4-yl]-amide or pharmaceutically acceptable salts, solvates, hydrates or morphological forms thereof for use in the treatment of pulmonary vascular disease and/or cardiac dysfunction in functional single ventricular heart disease patients, especially in Fontan-palliated patients. Moreover, the present invention relates to the use of high doses of macitentan for the manufacture of a medicament for the treatment of pulmonary vascular disease and/or cardiac dysfunction in functional single ventricular heart disease patients, especially in Fontan-palliated patients, as well as to a method for the treatment of pulmonary vascular disease and/or cardiac dysfunction in functional single ventricular heart disease patients, especially in Fontan-palliated patients comprising administering high doses of macitentan to a patient. Further, the present invention relates to a dosage regimen for the treatment of pulmonary vascular disease and/or cardiac dysfunction in functional single ventricular heart disease patients, especially in Fontan-palliated patients as well as to a combination of macitentan with one or more phosphodiesterase type 5 (PDE5) inhibitors, prostacyclin analogues, prostacyclin receptor agonists and/or soluble guanylate cyclase stimulators. Besides, the present invention relates to a pharmaceutical composition for the treatment of pulmonary vascular disease and/or cardiac dysfunction in functional single ventricular heart disease patients, especially in Fontan-palliated patients comprising a high dose of macitentan. Moreover, the present invention relates to the use of high doses of aprocitentan for the same purpose.

### BACKGROUND

The Fontan procedure is a palliative surgical procedure used in patients, typically children, with univentricular hearts or other related congenital heart diseases. It involves diverting the venous blood from the inferior vena cava (IVC) and superior vena cava (SVC) to the pulmonary arteries without passing through the morphologic right ventricle; i.e., the systemic and pulmonary circulations are placed in series with the functional single ventricle. The procedure was initially performed 1968 by Francis Fontan and Eugene Baudet. Contemporary modifications of surgical techniques have significantly improved survival. However, the resulting Fontan physiology is associated with high morbidity (Van der Ven et al, F1000 Research (2018), 7(F1000 Faculty Rev), 935). There is still a progressive risk of attrition and failure of the Fontan circulation over time, which lead to clinical worsening of these patients over time (Beghetti et al, Heart (2010), 96, 911-916).

With the improved outcome and the increasing number of procedures being performed, the number of surviving patients has risen and is expected to further rise substantially. Although life saving at the time of the operation, the Fontan procedure results in a pulmonary circulation that is very different from physiological conditions, with a progressive risk of attrition over time (Hosein et al., Eur. J. Cardiothorac. Surg. (2007), 31, 344-352). The number of patients likely to be affected is unknown, but the Fontan circulation may fail in many patients, leading to serious and potentially life-threatening sequalae (Gewillig, Heart (2005), 91, 839-846). Overall, the Fontan patients have decreased long-term survival, progressively deteriorating functional status and an increased risk of sudden death, which means that the procedure is considered to be palliative rather than curative (Fontan et al., Circulation (1990), 81, 1520-1536).

Aside from the impact of the Fontan physiology, the process of ageing itself has potentially important effects on the pulmonary vascular pathophysiology such as increasing pulmonary artery pressure (PAP) (Kovacs et al., Eur. Respir. J. (2009), 34, 888-894) and ventricular diastolic dysfunction. Preservation of the Fontan physiology and prevention of failure is therefore of critical importance, but no treatment has been demonstrated to be efficacious. Should the Fontan circulation fail, there are currently limited surgical or medical options and a lack of evidence-based guidelines to assist in management.

As the Fontan procedure was developed approximately three decades ago, the first patients are only now reaching their third and fourth decades of life and data in older patients with Fontan physiology will increasingly become available. Although the underlying pathophysiology of the failing Fontan is probably multifactorial, it is likely that a major underlying cause is alterations in, and damage to, the pulmonary vasculature.

Many post-Fontan complications arise as a result of increased venous pressure and congestion, and chronic low blood flow/cardiac output. Given the lack of ventricular force to drive blood flow through the pulmonary arteries, a low resistance (pulmonary vascular resistance [PVR]) and high capacitance system are mandatory for a well-functioning Fontan circuit. In the postoperative period, even small increases in PVR can lead to systemic venous hypertension associated with a decreased cardiac output despite a technically successful operation (Kirklin et al., Eur. J. Cardiothorac. Surg. (1990), 4, 2-7). In fact, a slight increase in PVR at any time to levels that would be readily tolerated in normal physiology may result in progressive failure of the Fontan circulation. Importantly, high PVR is a strong predictor of mortality (Griffiths et al., Ann. Thorac. Surg. (2009), 88, 558-563).

Prostacyclins have been rarely used in perioperative Fontan patients; beraprost reduces mean pulmonary artery pressure (PAP) and PVR in preoperative Fontan candidates with mild pulmonary hypertension, (Takahashi et al., Pediatr. Int (2003), 45, 671-675) and epoprostenol has been shown to prevent the rebound effect after inhaled nitric oxide (NO) cessation in the early postoperative phase (Miyaji et al., J. Thorac. Cardiovasc. Surg. (2003), 125, 437-439). When oral administration becomes possible, sildenafil is often used in the postoperative period as it is perceived as safe, with fast onset and good effectiveness. However, none of these medications (beraprost, epoprostenol, or sildenafil) are approved for these uses and there are no published data to confirm their efficacy or safety in this indication.

Bosentan has been shown to improve exercise capacity, functional class, health-related quality of life and haemodynamic parameters (including PVR and PAP) in patients with pulmonary arterial hypertension (PAH) (Gabbay et al., Vasc. Health Risk Manag. (2007), 3, 887-900; Valerio and Coghlan, Vasc. Health Risk Manag. (2009), 5, 607-619). Long-term treatment with bosentan improved symptoms and aortic oxygen saturation, functional class, maximal and submaximal exercise capacity, the Borg dyspnoea index, mean PAP, pulmonary blood flow and PVR in a patient with plastic bronchitis following Fontan (Apostolopoulou et al., J. Heart Lung Transplant. (2005), 24, 1174-1176; and Hebert et al., “The effect of Bosentan on exercise capacity in Fontan patients; rationale and design for the TEMPO study,” BMC Cardiovasc. Disord. 2013; 13: 36). The effect of long-term bosentan treatment on the liver is a potential concern for treating Fontan patients, especially in patients with hepatic complications. However, bosentan is not approved for these uses.

Given the important role of the pulmonary vascular circulation in Fontan physiology and the demonstrated increase in PAP with age, there is a major requirement for the development of effective and safe medical prevention or treatment for patients with worsening or failed Fontan system.

### SUMMARY OF THE INVENTION

It is an object of the present invention to provide a medication and/or treatment regimen for pulmonary vascular disease and/or cardiac dysfunction in functional single ventricular heart disease patients, especially in Fontan-palliated patients. In particular it is an object of the present invention to improve the oxygen uptake/consumption (peak VO₂) by the body. It is a further object of the present invention to reduce morbidity and/or mortality risk of pulmonary vascular disease and/or cardiac dysfunction in functional single ventricular heart disease patients, especially in Fontan-palliated patients. It is a further object of the present invention to provide a combination medication and/or treatment for pulmonary vascular disease and/or cardiac dysfunction in functional single ventricular heart disease patients, especially in Fontan-palliated patients.

### BRIEF DESCRIPTION OF THE FIGURES

FIG. 1 is a dose-response curve showing the change in hemoglobin (HGB) in function of the dose of macitentan administered to a human.

### DETAILED DESCRIPTION

The present inventors have recognized that, despite the potential occurrence of clinically relevant hemoglobin decreases, blood pressure decreases and/or edema or fluid retention increases, it is possible and safe and effective to treat pulmonary vascular disease and/or cardiac dysfunction in functional single ventricular heart disease patients, especially in Fontan-palliated patients with high doses of macitentan. In particular, it is possible to decrease the progress of pulmonary vascular disease and/or cardiac dysfunction, or even to improve the status of the pulmonary vascular disease and/or cardiac dysfunction.

In the present invention, macitentan is defined as propylsulfamic acid [5-(4-bromo-phenyl)-6-[2-(5-bromo-pyrimidin-2-yloxy)-ethoxy]-pyrimidin-4-yl]-amide, i.e. a compound of formula (I)

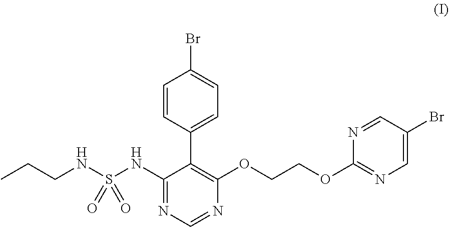 [SMILES: CCCNS(=O)(=O)Nc1ncnc(OCCOc2ncc(Br)cn2)c1-c1ccc(Br)cc1]

or a pharmaceutically acceptable salt, solvate, hydrate or morphological form thereof.

Macitentan is an endothelin receptor antagonist (ERA) that acts as an antagonist of two endothelin (ET) receptor subtypes, ET_{A} and ET_{B} (Kholdani et al, Macitentan for the treatment of pulmonary arterial hypertension. Vasc. Health Risk Manag. (2014), 10, 665-673).

Currently, macitentan is taken as a 10 mg oral dose once a day. Its half-life in humans is about 16 hours and steady state is reached by the third day of administration (Bruderer et al., Absorption, distribution, metabolism, and excretion of macitentan, a dual endothelin receptor antagonist, in humans. Xenobiotica (2012), 42(9), 901-910). It is absorbed slowly into the plasma (Sidharta et al., Macitentan: entry-into-humans study with a new endothelin receptor antagonist. Eur. J. Clin. Pharmacol. (2011), 67, 977-984). Macitentan dealkylates into the active metabolite ACT-132577, i.e. aprocitentan, which reaches its peak plasma concentration about 30 hours after the first dose is administered, and it has a half-life of approximately 48 hours. Although ACT-132577 has a lower affinity for the ET receptors than its parent compound (Iglarz et al., Pharmacology of macitentan, an orally active tissue-targeting dual endothelin receptor antagonist. J. Pharmacol. Exp. Ther. (2008), 327(3), 736-745), it maintains higher plasma concentrations than macitentan. Both compounds can be excreted from the body through the urine or feces.

In the following, several aspects of the invention will be explained.

(a) One aspect of the present invention relates to macitentan for use in the treatment of pulmonary vascular disease and/or cardiac dysfunction in functional single ventricular heart disease patients, especially in Fontan-palliated patients, wherein the dosage of macitentan is from 20 mg per day to 300 mg per day. The dosage may also be more than 20 mg per day to 300 mg per day. The dosage may be 25 mg per day to 300 mg per day. For example, the dosage is equal to or more than 20 mg per day to 250 mg per day. Preferably, the dosage is 25 mg to 200 mg per day.

According to a more preferred aspect, these dosages are applied once a day.

Currently, macitentan is administered as 10 mg oral dose once a day. However, with the present invention it is suggested to administer a high dosage of macitentan in order to improve the disease status, prevent the occurrence of disease worsening, and improve long-term survival and/or decrease hospitalization rate. Advantageously, if for instance compared to bosentan, there are fewer to no side effects on liver function and no or insignificant hepatotoxicity.

Further advantages are no further clinically relevant decrease in hemoglobin, no further clinically relevant decrease in blood pressure and/or no further clinically relevant increase in edema/fluid retention.

(b) Another aspect of the present invention relates to macitentan for use in the treatment of pulmonary vascular disease and/or cardiac dysfunction in functional single ventricular heart disease patients, especially in Fontan-palliated patients according to aspect (a), wherein the treatment means the reduction of morbidity and/or mortality risk of pulmonary vascular disease and/or cardiac dysfunction in functional single ventricular heart disease patients, especially in Fontan-palliated patients.

The reduction of morbidity and/or mortality risk of pulmonary vascular disease and/or cardiac dysfunction in functional single ventricular heart disease patients, especially in Fontan-palliated patients may be evaluated by cardiopulmonary exercise testing (CPET).

Exercise intolerance, indicated by dyspnea and fatigue during exertion, is a cardinal manifestation of heart failure (HF) (R. Malhotra et al., Cardiopulmonary Exercise Testing in Heart Failure, JACC: HEART FAILURE VOL. 4, NO. 8, pp. 607-616, 2016, which is considered as fully incorporated herein by reference).

Cardiopulmonary exercise testing (CPET) precisely defines maximum exercise capacity through measurement of peak oxygen uptake (VO₂). Cardiopulmonary exercise testing (CPET) provides breath-by-breath gas exchange measures of 3 variables: O₂ uptake (VO₂), carbon dioxide output (VCO₂), and ventilation (VE).

Measured VO₂ during a maximal symptom-limited CPET is the most objective method to assess functional capacity and consists of the following components (Balady G J et al., Clinician's Guide to cardiopulmonary exercise testing in adults: a scientific statement from the American Heart Association. Circulation 2010; 122: 191-225, which is considered as fully incorporated herein by reference):

Peak VO₂=HR_{MAX}×SV_{MAX}×(CaO₂−CvO₂)_{MAX}

where SV is stroke volume, and (CaO₂−CvO₂) is the net oxygen extraction of the peripheral tissues and is dependent on the hemoglobin concentration. VO₂ values are expressed in ml/kg/min

Furthermore, improvements in morbidity and/or mortality risk of pulmonary vascular disease and/or cardiac dysfunction in functional single ventricular heart disease patients, especially in Fontan-palliated patients may be demonstrated by daily activity as measured by an accelerometry measure (eg actigraphy); or 6-minute walk test distance; or quality of life questionnaire.

(c) Another aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is 60 to 90 mg per day. Preferably, the dosage is 65 to 85 mg per day, more preferably the dosage is 70 to 80 mg per day and most preferably the dosage is 75 mg per day. It is to be understood that each of the lower limits disclosed above may be combined with each of the upper limits, i.e. the dosage could also be from 60 to 85 mg, from 60 to 80 mg, or from 60 to 75 mg per day. Also disclosed are dosages from 65 to 90 mg, or 65 to 75 mg per day. Further preferred ranges are 72 to 78 mg per day. According to a more preferred aspect, these dosages are applied once a day.

(d) A further aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is 25 to 50 mg per day. Preferably, the dosage is 30 to 45 mg per day, more preferably 35 to 40 mg per day and most preferably 37.5 mg per day. It is to be understood that each of the lower limits disclosed above may be combined with each of the upper limits, i.e. the dosage could also be from 25 to 45 mg per day, or from 25 to 40 mg per day. Also disclosed are doses from 30 to 50 mg, or 30 to 40 mg per day. Further preferred ranges are 36 to 39 mg per day. According to a more preferred aspect, these dosages are applied once a day.

(e) A further aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is 110 to 200 mg per day. Preferably, the dosage is 125 to 175 mg per day, more preferably 140 to 160 mg per day and most preferably 150 mg per day. It is to be understood that each of the lower limits disclosed above may be combined with each of the upper limits, i.e. the dosage could also be from 110 to 175 mg per day, from 110 to 160 mg per day or from 110 to 150 mg per day. Also disclosed are doses from 125 to 160 mg or 140 to 175 mg per day. Further preferred ranges are 145 to 155 mg per day. According to a more preferred aspect, these dosages are applied once a day.

(f) A further aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is 60 to 90 mg twice per day. Preferably, the dosage is 65 to 85 mg twice per day, more preferably 70 to 80 mg twice per day and most preferably 75 mg twice per day. It is to be understood that each of the lower limits disclosed above may be combined with each of the upper limits, i.e. the dosage could also be from 60 to 85 mg twice per day, from 60 to 80 mg twice per day, or from 60 to 75 mg twice per day. Also disclosed are dosages from 65 to 90 mg twice per day, or 65 to 75 mg twice per day. Further preferred ranges are 72 to 78 mg twice per day.

(g) A further aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is 25 to 50 mg twice per day, preferably 30 to 45 mg twice per day, more preferably 35 to 40 mg twice per day and most preferably 37.5 mg twice per day. It is to be understood that each of the lower limits disclosed above may be combined with each of the upper limits, i.e. the dosage could also be from 25 to 45 mg twice per day, or from 25 to 40 mg twice per day. Also disclosed are doses from 30 to 50 mg twice per day, or 30 to 40 mg twice per day. Further preferred ranges are 36 to 39 mg twice per day.

(h) A further aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is escalated from 10 mg per day, followed by 25 to 50 mg per day, preferably 37.5 mg per day, and optionally followed by 60 to 90 mg per day, preferably 75 mg per day. Therein “followed by 25 to 50 mg per day” means preferably, the dosage is 30 to 45 mg per day, more preferably 35 to 40 mg per day and most preferably 37.5 mg per day. It is to be understood that each of the lower limits disclosed may be combined with each of the upper limits, i.e. the dosage could also be from 25 to 45 mg per day, or from 25 to 40 mg per day. Also disclosed are doses from 30 to 50 mg, or 30 to 40 mg per day. Further preferred ranges are 36 to 39 mg per day. It is to be understood that any escalation to a higher dose may be followed by return to the lower dose in case the higher dose is not tolerated.

Moreover, the phrase “optionally followed by 60 to 90 mg per day” means preferably, the dosage is 65 to 85 mg per day, more preferably the dosage is 70 to 80 mg per day and most preferably the dosage is 75 mg per day. It is to be understood that each of the lower limits disclosed above may be combined with each of the upper limits, i.e. the dosage could also be from 60 to 85 mg, from 60 to 80 mg, or from 60 to 75 mg per day. Also disclosed are dosages from 65 to 90 mg, or 65 to 75 mg per day. Further preferred ranges are 72 to 78 mg per day. It is to be understood that any escalation to a higher dose may be followed by return to the lower dose in case the higher dose is not tolerated.

According to a more preferred aspect, these dosages are applied once a day.

At present, the dosage of macitentan investigated in clinical trials for use in the treatment of pulmonary vascular disease and/or cardiac dysfunction in functional single ventricular heart disease patients, especially in Fontan-palliated patients is 10 mg per day. Patients obtaining this dosage may receive an immediate dose escalation to 37.5 mg per day, optionally followed by 75 mg per day.

A further aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is escalated from 10 mg per day, followed by 25 to 50 mg per day, preferably 37.5 mg per day, optionally followed by 60 to 90 mg per day, preferably 75 mg per day, optionally followed by 110 to 200 mg per day, preferably 150 mg per day.

The phrase “optionally followed by 110 to 200 mg per day” means preferably, the dosage is 125 to 175 mg per day, more preferably 140 to 160 mg per day and most preferably 150 mg per day. It is to be understood that each of the lower limits disclosed above may be combined with each of the upper limits, i.e. the dosage could also be from 110 to 175 mg per day, from 110 to 160 mg per day or from 110 to 150 mg per day. Also disclosed are doses from 125 to 160 mg or 140 to 175 mg per day. Further preferred ranges are 145 to 155 mg per day. According to a more preferred aspect, these dosages are applied once a day.

(i) A further aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is escalated from 10 mg per day, followed by 60 to 90 mg per day, preferably 75 mg once per day or 37.5 mg twice a day.

The phrase “optionally followed by 60 to 90 mg per day” means preferably, the dosage is 65 to 85 mg per day, more preferably the dosage is 70 to 80 mg per day and most preferably the dosage is 75 mg per day. It is to be understood that each of the lower limits disclosed above may be combined with each of the upper limits, i.e. the dosage could also be from 60 to 85 mg, from 60 to 80 mg, or from 60 to 75 mg per day. Also disclosed are dosages from 65 to 90 mg, or 65 to 75 mg per day. Further preferred ranges are 72 to 78 mg per day. It is to be understood that any escalation to a higher dose may be followed by return to the lower dose in case the higher dose is not tolerated.

According to a more preferred aspect, these dosages are applied once a day.

A further aspect of the present invention relates to macitentan for use in the treatment of pulmonary vascular disease and/or cardiac dysfunction in functional single ventricular heart disease patients, especially in Fontan-palliated patients according to aspect (a) or (b), wherein the dosage of macitentan is escalated from 10 mg per day, followed by 60 to 90 mg per day, preferably 75 mg once per day or 37.5 mg twice a day, optionally followed by 110 to 200 mg per day, preferably 150 mg per day.

The phrase “optionally followed by 110 to 200 mg per day” means preferably, the dosage is 125 to 175 mg per day, more preferably 140 to 160 mg per day and most preferably 150 mg per day. It is to be understood that each of the lower limits disclosed above may be combined with each of the upper limits, i.e. the dosage could also be from 110 to 175 mg per day, from 110 to 160 mg per day or from 110 to 150 mg per day. Also disclosed are doses from 125 to 160 mg or 140 to 175 mg per day. Further preferred ranges are 145 to 155 mg per day. According to a more preferred aspect, these dosages are applied once a day.

At present, the dosage of macitentan for use in the treatment of pulmonary vascular disease and/or cardiac dysfunction in functional single ventricular heart disease patients, especially in Fontan-palliated patients is 10 mg per day. Patients obtaining this dosage may receive an immediate dose escalation to 75 mg per day or to 150 mg per day.

(j) A further aspect of the present invention relates to macitentan for use in the treatment of pulmonary vascular disease and/or cardiac dysfunction in functional single ventricular heart disease patients, especially in Fontan-palliated patients according to aspect (h), wherein the dosage of macitentan is escalated from 10 mg once per day, preferably for 15 to 45 days; followed by 25 to 50 mg per day, preferably 37.5 mg once per day, preferably for 15 to 45 days; and optionally followed by 60 to 90 mg per day, preferably by 75 mg once per day or 37.5 mg twice per day. It is to be understood that any escalation to a higher dose may be followed by return to the lower dose in case the higher dose is not tolerated.

Therein, the term “15 to 45 days” means preferably 20 to 40 days, more preferably 21 to 35 days, and most preferably 28 to 30 days, i.e. about one month.

Moreover, the phrase “followed by 25 to 50 mg per day” means preferably, the dosage is 30 to 45 mg per day, more preferably 35 to 40 mg per day and most preferably 37.5 mg per day. It is to be understood that each of the lower limits disclosed may be combined with each of the upper limits, i.e. the dosage could also be from 25 to 45 mg per day, or from 25 to 40 mg per day. Also disclosed are doses from 30 to 50 mg, or 30 to 40 mg per day. Further preferred ranges are 36 to 39 mg per day.

Moreover, the phrase “followed by 60 to 90 mg per day” means preferably, the dosage is 65 to 85 mg per day, more preferably the dosage is 70 to 80 mg per day and most preferably the dosage is 75 mg per day. It is to be understood that each of the lower limits disclosed above may be combined with each of the upper limits, i.e. the dosage could also be from 60 to 85 mg, from 60 to 80 mg, or from 60 to 75 mg per day. Also disclosed are dosages from 65 to 90 mg, or 65 to 75 mg per day. Further preferred ranges are 72 to 78 mg per day, or 36 to 39 mg twice per day.

According to a more preferred aspect, these dosages are applied once a day.

A further aspect of the present invention relates to macitentan for use in the treatment of pulmonary vascular disease and/or cardiac dysfunction in functional single ventricular heart disease patients, especially in Fontan-palliated patients according to aspect (h), wherein the dosage of macitentan is escalated from 10 mg once per day, preferably for 15 to 45 days; followed by 25 to 50 mg per day, preferably 37.5 mg once per day, preferably for 15 to 45 days; optionally followed by 60 to 90 mg per day, preferably by 75 mg once per day or 37.5 mg twice per day, preferably for 15 to 45 days; optionally followed by 110 to 200 mg per day, preferably 150 mg per day.

The phrase “optionally followed by 110 to 200 mg per day” means preferably, the dosage is 125 to 175 mg per day, more preferably 140 to 160 mg per day and most preferably 150 mg per day. It is to be understood that each of the lower limits disclosed above may be combined with each of the upper limits, i.e. the dosage could also be from 110 to 175 mg per day, from 110 to 160 mg per day or from 110 to 150 mg per day. Also disclosed are doses from 125 to 160 mg or 140 to 175 mg per day. Further preferred ranges are 145 to 155 mg per day. According to a more preferred aspect, these dosages are applied once a day.

(k) A further aspect of the present invention relates to macitentan for use in the treatment of pulmonary vascular disease and/or cardiac dysfunction in functional single ventricular heart disease patients, especially in Fontan-palliated patients according to aspect (i), wherein the dosage of macitentan is escalated from 10 mg once per day, preferably for 15 to 45 days; followed by 60 to 90 mg per day, preferably by 75 mg once per day or 37.5 mg twice per day.

Therein, the term “15 to 45 days” means preferably 20 to 40 days, more preferably 21 to 35 days, and most preferably 28 to 30 days, i.e. about one month.

The phrase “followed by 60 to 90 mg per day” means preferably, the dosage is 65 to 85 mg per day, more preferably the dosage is 70 to 80 mg per day and most preferably the dosage is 75 mg per day. It is to be understood that each of the lower limits disclosed above may be combined with each of the upper limits, i.e. the dosage could also be from 60 to 85 mg, from 60 to 80 mg, or from 60 to 75 mg per day. Also disclosed are dosages from 65 to 90 mg, or 65 to 75 mg per day. Further preferred ranges are 72 to 78 mg per day, or 36 to 39 mg twice per day.

A further aspect of the present invention relates to macitentan for use in the treatment of pulmonary vascular disease and/or cardiac dysfunction in functional single ventricular heart disease patients, especially in Fontan-palliated patients according to (i), wherein the dosage of macitentan is escalated from 10 mg once per day, preferably for 15 to 45 days; followed by 60 to 90 mg per day, preferably by 75 mg once per day or 37.5 mg twice per day, preferably for 15 to 45 days; optionally followed by 110 to 200 mg per day, preferably 150 mg per day. The phrase “optionally followed by 110 to 200 mg per day” means preferably, the dosage is 125 to 175 mg per day, more preferably 140 to 160 mg per day and most preferably 150 mg per day. It is to be understood that each of the lower limits disclosed above may be combined with each of the upper limits, i.e. the dosage could also be from 110 to 175 mg per day, from 110 to 160 mg per day or from 110 to 150 mg per day. Also disclosed are doses from 125 to 160 mg or 140 to 175 mg per day. Further preferred ranges are 145 to 155 mg per day.

According to a more preferred aspect, these dosages are applied once a day.

(l) A further aspect of the present invention relates to macitentan for use in the treatment of pulmonary vascular disease and/or cardiac dysfunction in functional single ventricular heart disease patients, especially in Fontan-palliated patients according to any one of aspects (a) to (d), wherein the dosage of macitentan is escalated to 25 to 50 mg per day, preferably 37.5 mg per day; preferably for 15 to 45 days; optionally followed by 60 to 90 mg per day, preferably by 75 mg per day; provided that the patient is already treated with an endothelin receptor antagonist preferably selected from bosentan and ambrisentan. This means that a patient has already been treated or is treated with an endothelin receptor antagonist. It is to be understood that any escalation to a higher dose may be followed by return to the lower dose in case the higher dose is not tolerated.

Therein, the term “15 to 45 days” means preferably 20 to 40 days, more preferably 21 to 35 days, and most preferably 28 to 30 days, i.e. about one month.

Moreover, the phrase “followed by 25 to 50 mg per day” means preferably, the dosage is 30 to 45 mg per day, more preferably 35 to 40 mg per day and most preferably 37.5 mg per day. It is to be understood that each of the lower limits disclosed may be combined with each of the upper limits, i.e. the dosage could also be from 25 to 45 mg per day, or from 25 to 40 mg per day. Also disclosed are doses from 30 to 50 mg, or 30 to 40 mg per day. Further preferred ranges are 36 to 39 mg per day.

Moreover, the phrase “followed by 60 to 90 mg per day” means preferably, the dosage is 65 to 85 mg per day, more preferably the dosage is 70 to 80 mg per day and most preferably the dosage is 75 mg per day. It is to be understood that each of the lower limits disclosed above may be combined with each of the upper limits, i.e. the dosage could also be from 60 to 85 mg, from 60 to 80 mg, or from 60 to 75 mg per day. Also disclosed are dosages from 65 to 90 mg, or 65 to 75 mg per day. Further preferred ranges are 72 to 78 mg per day, or 36 to 39 mg twice per day.

It is to be understood that, optionally, the dosage of macitentan can be further raised to reach from 110 to 200 mg per day. The dosage is thereby escalated as in aspect (i), (j) or (k).

According to a more preferred aspect, these dosages are applied once a day.

(m) A further aspect of the present invention relates to macitentan for use in the treatment of pulmonary vascular disease and/or cardiac dysfunction in functional single ventricular heart disease patients, especially in Fontan-palliated patients according to any one of aspects (a) to (c), wherein the dosage of macitentan is 60 to 90 mg per day, preferably 75 mg per day; provided that the patient is already treated with an endothelin receptor antagonist preferably selected from bosentan, and ambrisentan.

The phrase “60 to 90 mg per day” means preferably, the dosage is 65 to 85 mg per day, more preferably the dosage is 70 to 80 mg per day and most preferably the dosage is 75 mg per day. It is to be understood that each of the lower limits disclosed above may be combined with each of the upper limits, i.e. the dosage could also be from 60 to 85 mg, from 60 to 80 mg, or from 60 to 75 mg per day. Also disclosed are dosages from 65 to 90 mg, or 65 to 75 mg per day. Further preferred ranges are 72 to 78 mg per day, or 36 to 39 mg twice per day.

It is to be understood, that optionally, the dosage of macitentan can be further raised to reach from 110 to 200 mg per day. The dosage is thereby escalated as in aspect (i), (j) or (k).

According to a more preferred aspect, these dosages are applied once a day.

(n) A further aspect of the present invention relates to macitentan for use in the treatment of pulmonary vascular disease and/or cardiac dysfunction in functional single ventricular heart disease patients, especially in Fontan-palliated patients according to any one of aspects (a) to (m), wherein macitentan is combined with a PDE5 inhibitor and/or a prostacyclin analogue, and/or a prostacyclin receptor agonist and/or a soluble guanylate cyclase stimulator.

Inhibition of the cyclic guanosine monophosphate (cGMP) degrading enzyme phosphodiesterase type 5 results in vasodilation through the NO/cGMP pathway at sites expressing this enzyme. Since the pulmonary vasculature contains substantial amounts of phosphodiesterase type 5, the potential clinical benefit of phosphodiesterase type 5 inhibitors (PDE5 is) has been investigated in PAH. In addition, PDE5 is exert antiproliferative effects (ESC/ERS Guidelines; European Heart Journal (2016), 37, 67-119).

Prostacyclin is produced predominantly by endothelial cells and induces potent vasodilation of all vascular beds. This compound is the most potent endogenous inhibitor of platelet aggregation and also appears to have both cytoprotective and antiproliferative activities. Dysregulation of the prostacyclin metabolic pathways has been shown in patients with PAH as assessed by a reduction of prostacyclin synthase expression in the pulmonary arteries and of prostacyclin urinary metabolites. The clinical use of prostacyclin in patients with PAH has been extended by the synthesis of stable analogues that possess different pharmacokinetic properties but share qualitatively similar pharmacodynamic effects (ESC/ERS Guidelines; European Heart Journal (2016), 37, 67-119).

No Drug-Drug Interaction has been observed for macitentan and its active metabolite, ACT-132577 so far.

For example, macitentan 10 mg per day o.d. has not shown any effect on the pharmacokinetics of 1 mg rosuvastatin, which suggests that BCRP transporters have not been inhibited. BCRP is an efflux pump located in the gut, liver canalicular membrane, and kidney, and is exposed to intracellular drug concentrations in the liver and the kidney.

Macitentan and ACT-132577 activated human PXR with EC₅₀ values of 1.1 to 1.2 μM and 7.2 to 8.7 μM, respectively. In human hepatocytes, both compounds elicited concentration-dependent increases in CYP3A4 mRNA and enzyme activity.

Predicted peak plasma concentrations of macitentan and ACT-132577 in PAH patients at 75 mg per day dose are expected to be around 5 μM and 14 μM, respectively, based on the PK Sub-study and assuming dose linearity. Taking into account the high degree of protein binding, free plasma concentrations are expected to be in the range of 0.02 μM to 0.07 μM for macitentan and ACT-132577, respectively. It is not likely that these unbound concentrations of macitentan and ACT-132577 result in any inhibition of BCRP in the liver or kidney or induction of CYP3A4 enzyme in the liver.

Therefore, the dosage of macitentan may be 60 to 90 mg per day in aspect (n). Preferably, the dosage is 65 to 85 mg per day, more preferably the dosage is 70 to 80 mg per day and most preferably the dosage is 75 mg per day. It is to be understood that each of the lower limits disclosed above may be combined with each of the upper limits, i.e. the dosage could also be from 60 to 85 mg, from 60 to 80 mg, or from 60 to 75 mg per day. Also disclosed are dosages from 65 to 90 mg, or 65 to 75 mg per day. A further preferred dosage range is 72 to 78 mg per day.

Further, the dosage of macitentan may be escalated from 10 mg per day, followed by 25 to 50 mg per day, preferably 37.5 mg per day, followed by 60 to 90 mg per day, preferably 75 mg per day, optionally followed by 100 to 200 mg per day, preferably 150 mg per day.

(o) A further aspect of the present invention relates to macitentan for use in the treatment of pulmonary vascular disease and/or cardiac dysfunction in functional single ventricular heart disease patients, especially in Fontan-palliated patients according to aspect (n), wherein the PDE5 inhibitor is selected from sildenafil, tadalafil, vardenafil, and udenafil; the prostacyclin analogue is selected from epoprostenol, treprostinil, iloprost, and beraprost; the prostacyclin receptor agonist is selected from selexipag and ralinepag; and the soluble guanylate cyclase stimulator is selected from riociguat and vericiguat.

Therein, macitentan has a dosage or dosage regimen according to any one of aspects (a) to (l) and (n).

(p) A further aspect of the present invention relates to macitentan for use in the treatment of pulmonary vascular disease and/or cardiac dysfunction in functional single ventricular heart disease patients, especially in Fontan-palliated patients according to aspect (n) or (o), wherein macitentan is combined with tadalafil and/or selexipag or ralinepag. Alternatively, macitentan is combined with udenafil and/or selexipag or ralinepag. Preferably, macitentan is combined with tadalafil and/or selexipag. Alternatively, macitentan is combined with udenafil and/or selexipag.

Therein, macitentan has a dosage or dosage regimen according to any one of aspects (a) to (k) and (n).

(q) A further aspect of the present invention relates to macitentan for use in the treatment of pulmonary vascular disease and/or cardiac dysfunction in functional single ventricular heart disease patients, especially in Fontan-palliated patients according to aspect (p), wherein tadalafil, if applicable, has a dose of 20 to 40 mg per day, preferably 40 mg per day, udenafil, if applicable, has a dose of 5 to 200 mg per day, preferably 100 to 200 mg per day, preferably 150 to 200 mg, preferably 175 mg per day, preferably 87.5 mg twice per day, preferably 43.5 mg four times per day, preferably 58 mg thrice per day, selexipag, if applicable, has a dose of 0.2 to 1.6 mg twice per day, and ralinepag, if applicable, has a dose of 0.05 to 1.45 mg per day.

Therein, macitentan has a dosage or dosage regimen according to any one of aspects (a) to (k) and (n).

(r) A further aspect of the present invention relates to a pharmaceutical composition for use in the treatment of pulmonary vascular disease and/or cardiac dysfunction in functional single ventricular heart disease patients, especially in Fontan-palliated patients comprising macitentan and at least a pharmaceutically acceptable excipient, containing macitentan in an amount 20 mg to 300 mg, for example, more than 20 mg to 300 mg, for example more than 20 mg to 250 mg, for example 25 mg to 200 mg, preferably 37.5 mg, 75 mg or 150 mg, more preferably 37.5 mg or 75 mg, most preferably 75 mg.

It is to be understood that macitentan may have a dosage according to any one of the daily dosages of aspects (a) to (e).

(s) A further aspect of the present invention relates to the pharmaceutical composition according to aspect (r), which comprises
i) macitentan in a total amount of 10 to 50% in weight based on the total weight of the pharmaceutical composition,
ii) a filler, consisting of lactose monohydrate with microcrystalline cellulose, in a total amount of 10 to 85% in weight based on the total weight of the pharmaceutical composition,
iii) a disintegrant, consisting of sodium starch glycolate or a combination of sodium starch glycolate and polyvinylpyrrolidone, in a total amount of 1 to 10% in weight based on the total weight of the pharmaceutical composition,
iv) a surfactant, consisting of a polysorbate, in a total amount of 0.1 to 1% in weight based on the total weight of the pharmaceutical composition, and
v) a lubricant, consisting of magnesium stearate, in a total amount of 0.05 to 5% in weight based on the total weight of the pharmaceutical composition.

(t) A further aspect of the present invention relates to the pharmaceutical composition according to aspect (r) or (s), which is in the form of a capsule or a tablet (in particular in the form of a tablet, notably a tablet containing 75 mg of macitentan).

In the following, the invention is described in the format of macitentan for the manufacture of a medicament for use in the treatment of pulmonary vascular disease and/or cardiac dysfunction in functional single ventricular heart disease patients, especially in Fontan-palliated patients:

(a′) One aspect of the present invention relates to macitentan for the manufacture of a medicament for use in the treatment of pulmonary vascular disease and/or cardiac dysfunction in functional single ventricular heart disease patients, especially in Fontan-palliated patients, wherein the dosage of macitentan is from 20 mg per day to 300 mg per day. It is to be understood that the disclosure of aspect (a) applies analogously.

(b′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (a′), wherein the treatment means the reduction of morbidity and/or mortality risk of pulmonary vascular disease and/or cardiac dysfunction in functional single ventricular heart disease patients, especially in Fontan-palliated patients. It is to be understood that the disclosure of aspect (b) applies analogously.

(c′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (a′) or (b′), wherein the dosage of macitentan is 60 to 90 mg per day, preferably 65 to 85 mg per day, more preferably 70 to 80 mg per day and most preferably 75 mg per day. It is to be understood that the disclosure of aspect (c) applies analogously.

(d′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (a′) or (b′), wherein the dosage of macitentan is 25 to 50 mg per day, preferably 30 to 45 mg per day, more preferably 35 to 40 mg per day and most preferably 37.5 mg per day. It is to be understood that the disclosure of aspect (d) applies analogously.

(e′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (a′) or (b′), wherein the dosage of macitentan is 110 to 200 mg per day, preferably 125 to 175 mg per day, more preferably 140 to 160 mg per day and most preferably 150 mg per day. It is to be understood that the disclosure of aspect (e) applies analogously.

(f′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (a′) or (b′), wherein the dosage of macitentan is 60 to 90 mg twice per day, preferably 65 to 85 mg twice per day, more preferably 70 to 80 mg twice per day and most preferably 75 mg twice per day. It is to be understood that the disclosure of aspect (f) applies analogously.

(g′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (a′) or (b′), wherein the dosage of macitentan is 25 to 50 mg twice per day, preferably 30 to 45 mg twice per day, more preferably 35 to 40 mg twice per day and most preferably 37.5 mg twice per day. It is to be understood that the disclosure of aspect (g) applies analogously.

(h′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (a′) or (b′), wherein the dosage of macitentan is escalated from 10 mg per day, followed by 25 to 50 mg per day, preferably 37.5 mg per day, and optionally followed by 60 to 90 mg per day, preferably 75 mg per day. It is to be understood that the disclosure of aspect (h) applies analogously.

(i′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (a) or (b), wherein the dosage of macitentan is escalated from 10 mg per day, followed by 60 to 90 mg per day, preferably 75 mg once per day or 37.5 mg twice a day. It is to be understood that the disclosure of aspect (i) applies analogously.

(j′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (h′), wherein the dosage of macitentan is escalated from 10 mg once per day, preferably for 15 to 45 days; followed by 25 to 50 mg per day, preferably 37.5 mg once per day, preferably for 15 to 45 days; and optionally followed by 60 to 90 mg per day, preferably by 75 mg once per day or 37.5 mg twice per day. It is to be understood that the disclosure of aspect (j) applies analogously.

(k′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (i′), wherein the dosage of macitentan is escalated from 10 mg once per day, preferably for 15 to 45 days; followed by 60 to 90 mg per day, preferably by 75 mg once per day or 37.5 mg twice per day. It is to be understood that the disclosure of aspect (k) applies analogously.

(l′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to any one of aspects (a′) to (d′), wherein the dosage of macitentan is escalated from 25 to 50 mg per day, preferably 37.5 mg per day, preferably for 15 to 45 days; optionally followed by 60 to 90 mg per day, preferably by 75 mg per day; provided that the patient is already treated with an endothelin receptor antagonist preferably selected from bosentan and ambrisentan. It is to be understood that the disclosure of aspect (l) applies analogously.

(m′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to any one of aspects (a′) to (c′), wherein the dosage of macitentan is 60 to 90 mg per day, preferably 75 mg per day; provided that the patient is already treated with an endothelin receptor antagonist preferably selected from bosentan and ambrisentan. It is to be understood that the disclosure of aspect (m) applies analogously.

(n′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to any one of aspects (a′) to (m′), wherein macitentan is combined with a PDE5 inhibitor and/or a prostacyclin analogue, and/or a prostacyclin receptor agonist and/or a soluble guanylate cyclase stimulator. It is to be understood that the disclosure of aspect (n) applies analogously.

(o′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (n′), wherein the PDE5 inhibitor is selected from sildenafil, tadalafil, vardenafil, and udenafil; the prostacyclin analogue is selected from epoprostenol, treprostinil, iloprost, and beraprost; the prostacyclin receptor agonist is selected from selexipag and ralinepag; and the soluble guanylate cyclase stimulator is selected from riociguat and vericiguat. It is to be understood that the disclosure of aspect (o) applies analogously.

(p′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (n′) or (o′), wherein macitentan is combined with tadalafil and/or selexipag or ralinepag, preferably tadalafil and/or selexipag. Alternatively, macitentan is combined with udenafil and/or selexipag or ralinepag, preferably udenafil and/or selexipag. It is to be understood that the disclosure of aspect (p) applies analogously.

(q′) Another aspect of the present invention relates to macitentan for the manufacture of a medicament for use in the treatment of pulmonary vascular disease and/or cardiac dysfunction in functional single ventricular heart disease patients, especially in Fontan-palliated patients according to aspect (p′), wherein tadalafil, if applicable, has a dose of 20 to 40 mg per day, preferably 40 mg per day, udenafil, if applicable, has a dose of 5 to 200 mg per day, preferably 100 to 200 mg per day, preferably 150 to 200 mg, preferably 175 mg per day, preferably 87.5 mg twice per day, preferably 43.5 mg four times per day, preferably 58 mg thrice per day, selexipag, if applicable, has a dose of 0.2 to 1.6 mg twice per day and ralinepag, if applicable, has a dose of 0.05 to 1.45 mg per day. It is to be understood that the disclosure of aspect (q) applies analogously.

It is to be understood that the comments and details of aspects (a) to (q) also apply to aspects (a′) to (q′).

Moreover, it is to be understood that all disclosed aspects (a) to (q) are to be regarded as disclosed also in the form of a method of treatment:

In the following, the invention is described in the format of a method of treatment of pulmonary vascular disease and/or cardiac dysfunction in functional single ventricular heart disease patients, especially in Fontan-palliated patients:

(a″) One aspect of the present invention relates to a method of treatment of pulmonary vascular disease and/or cardiac dysfunction in functional single ventricular heart disease patients, especially in Fontan-palliated patients, said method comprising administering macitentan to said patients in a dosage of 20 mg per day to 300 mg per day. It is to be understood that the disclosure of aspect (a) applies analogously.

(b″) Another aspect of the present invention relates to a method of treatment of pulmonary vascular disease and/or cardiac dysfunction in functional single ventricular heart disease patients, especially in Fontan-palliated patients according to aspect (a″), wherein the treatment means the reduction of morbidity and/or mortality risk of pulmonary vascular disease and/or cardiac dysfunction in functional single ventricular heart disease patients, especially in Fontan-palliated patients. It is to be understood that the disclosure of aspect (b) applies analogously.

(c″) Another aspect of the present invention relates to a method of treatment of pulmonary vascular disease and/or cardiac dysfunction in functional single ventricular heart disease patients, especially in Fontan-palliated patients according to aspect (a″) or (b″), wherein the dosage of macitentan is 60 to 90 mg per day, preferably 65 to 85 mg per day, more preferably 70 to 80 mg per day and most preferably 75 mg per day. It is to be understood that the disclosure of aspect (c) applies analogously.

(d″) Another aspect of the present invention relates to a method of treatment of pulmonary vascular disease and/or cardiac dysfunction in functional single ventricular heart disease patients, especially in Fontan-palliated patients according to aspect (a″) or (b″), wherein the dosage of macitentan is 25 to 50 mg per day, preferably 30 to 45 mg per day, more preferably 35 to 40 mg per day and most preferably 37.5 mg per day. It is to be understood that the disclosure of aspect (d) applies analogously.

(e″) Another aspect of the present invention relates to a method of treatment of pulmonary vascular disease and/or cardiac dysfunction in functional single ventricular heart disease patients, especially in Fontan-palliated patients according to aspect (a″) or (b″), wherein the dosage of macitentan is 110 to 200 mg per day, preferably 125 to 175 mg per day, more preferably 140 to 160 mg per day and most preferably 150 mg per day. It is to be understood that the disclosure of aspect (e) applies analogously.

(f″) Another aspect of the present invention relates to a method of treatment of pulmonary vascular disease and/or cardiac dysfunction in functional single ventricular heart disease patients, especially in Fontan-palliated patients according to aspect (a″) or (b″), wherein the dosage of macitentan is 60 to 90 mg twice per day, preferably 65 to 85 mg twice per day, more preferably 70 to 80 mg twice per day and most preferably 75 mg twice per day. It is to be understood that the disclosure of aspect (f) applies analogously.

(g″) Another aspect of the present invention relates to a method of treatment of pulmonary vascular disease and/or cardiac dysfunction in functional single ventricular heart disease patients, especially in Fontan-palliated patients according to aspect (a″) or (b″), wherein the dosage of macitentan is 25 to 50 mg twice per day, preferably 30 to 45 mg twice per day, more preferably 35 to 40 mg twice per day and most preferably 37.5 mg twice per day. It is to be understood that the disclosure of aspect (g) applies analogously.

(h″) Another aspect of the present invention relates to a method of treatment of pulmonary vascular disease and/or cardiac dysfunction in functional single ventricular heart disease patients, especially in Fontan-palliated patients according to aspect (a″) or (b″), wherein the dosage of macitentan is escalated from 10 mg per day, followed by 25 to 50 mg per day, preferably 37.5 mg per day, and optionally followed by 60 to 90 mg per day, preferably 75 mg per day. It is to be understood that the disclosure of aspect (h) applies analogously.

(i″) Another aspect of the present invention relates to a method of treatment of pulmonary vascular disease and/or cardiac dysfunction in functional single ventricular heart disease patients, especially in Fontan-palliated patients according to aspect (a″) or (b″), wherein the dosage of macitentan is escalated from 10 mg per day, followed by 60 to 90 mg per day, preferably 75 mg once per day or 37.5 mg twice a day. It is to be understood that the disclosure of aspect (i) applies analogously.

(j″) Another aspect of the present invention relates to a method of treatment of pulmonary vascular disease and/or cardiac dysfunction in functional single ventricular heart disease patients, especially in Fontan-palliated patients according to aspect (h″), wherein the dosage of macitentan is escalated from 10 mg once per day, preferably for 15 to 45 days; followed by 25 to 50 mg per day, preferably 37.5 mg once per day, preferably for 15 to 45 days; and optionally followed by 60 to 90 mg per day, preferably by 75 mg once per day or 37.5 mg twice per day. It is to be understood that the disclosure of aspect (j) applies analogously.

(k″) Another aspect of the present invention relates to a method of treatment of pulmonary vascular disease and/or cardiac dysfunction in functional single ventricular heart disease patients, especially in Fontan-palliated patients according to aspect (i″), wherein the dosage of macitentan is escalated from 10 mg once per day, preferably for 15 to 45 days; followed by 60 to 90 mg per day, preferably by 75 mg once per day or 37.5 mg twice per day. It is to be understood that the disclosure of aspect (k) applies analogously.

(l″) Another aspect of the present invention relates to a method of treatment of pulmonary vascular disease and/or cardiac dysfunction in functional single ventricular heart disease patients, especially in Fontan-palliated patients according to any one of aspects (a″) to (d″), wherein the dosage of macitentan is escalated from 25 to 50 mg per day, preferably 37.5 mg per day, preferably for 15 to 45 days; optionally followed by 60 to 90 mg per day, preferably by 75 mg per day; provided that the patient is already treated with an endothelin receptor antagonist preferably selected from bosentan and ambrisentan. It is to be understood that the disclosure of aspect (l) applies analogously.

(m″) Another aspect of the present invention relates to a method of treatment of pulmonary vascular disease and/or cardiac dysfunction in functional single ventricular heart disease patients, especially in Fontan-palliated patients according to any one of aspects (a″) to (c″), wherein the dosage of macitentan is 60 to 90 mg per day, preferably 75 mg per day; provided that the patient is already treated with an endothelin receptor antagonist preferably selected from bosentan and ambrisentan. It is to be understood that the disclosure of aspect (m) applies analogously.

(n″) Another aspect of the present invention relates to a method of treatment of pulmonary vascular disease and/or cardiac dysfunction in functional single ventricular heart disease patients, especially in Fontan-palliated patients according to any one of aspects (a″) to (m″), wherein macitentan is combined with a PDE5 inhibitor and/or a prostacyclin analogue, and/or a prostacyclin receptor agonist and/or a soluble guanylate cyclase stimulator. It is to be understood that the disclosure of aspect (n) applies analogously.

(o″) Another aspect of the present invention relates to a method of treatment of pulmonary vascular disease and/or cardiac dysfunction in functional single ventricular heart disease patients, especially in Fontan-palliated patients according to aspect (n″), wherein the PDE5 inhibitor is selected from sildenafil, tadalafil, vardenafil, and udenafil; the prostacyclin analogue is selected from epoprostenol, treprostinil, iloprost, and beraprost; the prostacyclin receptor agonist is selected from selexipag and ralinepag; and the soluble guanylate cyclase stimulator is selected from riociguat and vericiguat. It is to be understood that the disclosure of aspect (o) applies analogously.

(p″) Another aspect of the present invention relates to a method of treatment of pulmonary vascular disease and/or cardiac dysfunction in functional single ventricular heart disease patients, especially in Fontan-palliated patients according to aspect (n″) or (o″), wherein macitentan is combined with tadalafil and/or selexipag or ralinepag, preferably tadalafil and/or selexipag. Alternatively, macitentan is combined with udenafil and/or selexipag or ralinepag, preferably udenafil and/or selexipag. It is to be understood that the disclosure of aspect (p) applies analogously.

(q″) Another aspect of the present invention relates to a method of treatment of pulmonary vascular disease and/or cardiac dysfunction in functional single ventricular heart disease patients, especially in Fontan-palliated patients according to aspect (p″), wherein tadalafil, if applicable, has a dose of 20 to 40 mg per day, preferably 40 mg per day, udenafil, if applicable, has a dose of 5 to 200 mg per day, preferably 100 to 200 mg per day, preferably 150 to 200 mg, preferably 175 mg per day, preferably 87.5 mg twice per day, preferably 43.5 mg four times per day, preferably 58 mg thrice per day, selexipag, if applicable, has a dose of 0.2 to 1.6 mg twice per day and ralinepag, if applicable, has a dose of 0.05 to 1.45 mg per day. It is to be understood that the disclosure of aspect (q) applies analogously.

It is to be understood that the comments and details of aspects (a) to (q) also apply to aspects (a″) to (q″).

According to a further aspect of the present invention, in each of the above-mentioned aspects, that is, in each of aspects (a) to (r) and (t), (a′) to (q′) as well as (a″) to (q″), macitentan can be replaced by its active metabolite, known under the code name ACT-132577 and the International Non-proprietary Name aprocitentan, which has the chemical formula

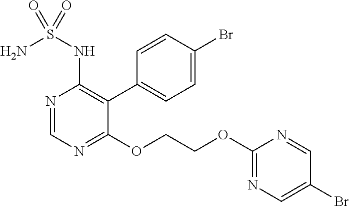 [SMILES: NS(=O)(=O)Nc1ncnc(OCCOc2ncc(Br)cn2)c1-c1ccc(Br)cc1]

whereby any weight amount of macitentan will be replaced a 5-fold weight amount of aprocitentan.

For example, taking aspect (c) of the invention described above, a further aspect of the invention relates to aprocitentan for use in the treatment of pulmonary vascular disease and/or cardiac dysfunction in functional single ventricular heart disease patients, especially in Fontan-palliated patients according to aspect (a) or (b) wherein the weight amounts of macitentan will be replaced a 5-fold weight amount of aprocitentan, wherein the dosage of aprocitentan is 300 to 450 mg per day. Preferably, the dosage of aprocitentan is 325 to 425 mg per day, more preferably the dosage of aprocitentan is 350 to 400 mg per day and most preferably the dosage of aprocitentan is 375 mg per day. It is to be understood that each of the lower limits disclosed above may be combined with each of the upper limits, i.e. the dosage of aprocitentan could also be from 300 to 425 mg, from 300 to 400 mg, or from 300 to 375 mg per day. Also disclosed are dosages of aprocitentan from 325 to 450 mg, or 325 to 375 mg per day. Further preferred ranges are 360 to 390 mg of aprocitentan per day. According to a more preferred aspect, these dosages of aprocitentan are applied once a day.

It may be stated that relevant portions of the cited reference documents are each incorporated herein by reference.

Moreover, the following abbreviations are used throughout the present specification.

### Abbreviations

AE adverse event
ALT alanine aminotransferase
AST aspartate aminotransferase
BCRP breast cancer resistance protein
BMI body mass index
bpm beats per minute
CI cardiac index
CYP cytochrome P450
ECG electrocardiogram
eGFR estimated glomerular filtration rate

### EOS End-of-Study

### EOT End-of-Treatment

ERA endothelin receptor antagonist
ET endothelin
HR heart rate

### IB Investigator's Brochure

IVC inferior vena cava

### MDRD Modification of Diet in Renal Disease

PAH pulmonary arterial hypertension
PAP pulmonary artery pressure
PD pharmacodynamics
PDE5 cyclic guanosine 3′,5′-monophosphate (cGMP) phosphodiesterase type 5
PDE5 is PDE5 inhibitors
PH pulmonary hypertension
PK pharmacokinetics
PVR pulmonary vascular resistance
qd once daily
QTcB QT interval corrected according to Bazett's formula
QTcF QT interval corrected according to Fridericia's formula
RBC red blood cell
SVC superior vena cava
tid 3 times daily
WBC white blood cell

### EXPERIMENTAL PART

The following non-limitative examples illustrate the invention.

### EXAMPLES

### Example 1: Effect of Macitentan on Decrease in Hemoglobin Concentration

Hemoglobin measurements were pooled from 3 Phase I clinical studies in healthy volunteers:
Study AC-055-102: Investigation of the PK, PD, safety and tolerability of macitentan in male subjects (the study protocol is described in the following publication: Sidharta et al., Safety, tolerability, pharmacokinetics, and pharmacodynamics of macitentan, an endothelin receptor antagonist, in an ascending multiple-dose study in healthy subjects. J. Clin. Pharmacol. (2013), 53(11), 1131-1138)
Study AC-055-116: Investigation of the PK, PD, safety and tolerability of macitentan in male Japanese subjects (the study protocol is described in the following publication: Yokoyama et al., Tolerability, Safety, Pharmacokinetics, and Pharmacodynamics of Macitentan, a New Endothelin Receptor Antagonist, in Healthy Japanese Male Subjects. Rinsho yakuri/Japanese Journal of Clinical Pharmacology and Therapeutics (2016), 47, 143-150)
Study AC-055-117: Investigation of the PK, PD, safety and tolerability of macitentan in male Korean subjects (the study protocol is described in the following publication: Ahn et al., Pharmacokinetic-pharmacodynamic relationships of macitentan, a new endothelin receptor antagonist, after multiple dosing in healthy Korean subjects, Am. J. Cardiovasc. Drugs (2014), 14(5), 377-385)

Hemoglobin concentrations measured in the morning of Day 11 of macitentan treatment and at baseline on Day −1 were used in the analysis. Changes in hemoglobin concentrations compared to baseline were regressed against the different dose levels of macitentan, including placebo.

An Emax curve with baseline was fitted and the following parameters were estimated by nonlinear regression:
E0: Change in hemoglobin without macitentan
Emax: Maximum change in hemoglobin theoretically could be elicited by macitentan
ED50: the dose resulting in 50% reduction of hemoglobin

The following formula was used:

Change in hemoglobin=E0+((Macitentan dose×E max)/(Macitentan dose+ED50))

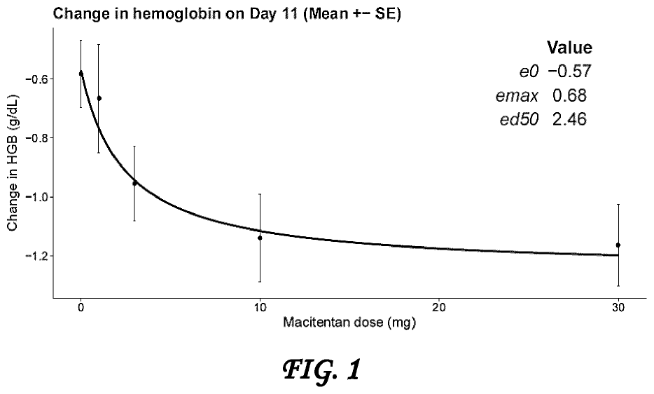

The resulting dose-response curve is shown in FIG. 1.

Based on the analysis, the maximum effect of macitentan on hemoglobin decrease would be around 1.23 g/dL and the effect of macitentan on hemoglobin decrease plateaus already at the 10 mg dose (see FIG. 1). Therefore, no clinically relevant decrease in hemoglobin is expected above a 10 mg dose of macitentan in humans.

### Example 2: Administration of High Doses of Macitentan

This is a single-sequence, open-label, single-center, Phase 1 study in healthy male subjects. The study assessed the effect of 75 mg qd administration of macitentan. The study includes a screening phase of 21 days before the first macitentan administration, followed by an open-label treatment phase. Randomization was not used and subjects received the same treatment.

A. Subject Population

29 subjects were enrolled. 11 subjects were enrolled under an initial protocol that did not include up-titration of macitentan treatment; 18 subjects were enrolled under an amended protocol that included up-titration of macitentan treatment.

B. Study Treatment

B.1. Study Treatment without Uptitration of Macitentan

Administration of 75 mg qd macitentan started on Day 1. Macitentan was administered every day in the morning between 8:00 and 11:00 with 240 mL of noncarbonated water. One 75-mg macitentan film-coated tablet was thus administered as multiple qd oral doses under fed conditions.

Subjects received standardized meals and on dosing days when macitentan was administered in fed state, at the following time points:
Evening meal not later than 10 hours before macitentan administration next day.
Breakfast within 30 minutes prior to macitentan administration (Days 1-9 and Days 11-12).
Lunch approximately 4 hours after macitentan administration.
Snack approximately 7 hours after macitentan administration.
Evening meal approximately 10 hours after macitentan administration.

Subjects were planned to take macitentan 75 mg for 15 days. However the study was interrupted on the morning of day 8, i.e. when subjects had received 7 doses of macitentan 75 mg.

B.2. Study Treatment Including Uptitration of Macitentan

Macitentan was administered in an up-titration regimen in the morning between 8:00 and 11:00 with 240 mL of noncarbonated water. One macitentan film-coated tablet (10 mg, 37.5 mg or 75 mg) was administered as multiple qd oral doses under fed conditions on Days 1-9 and Days 11-12, and under fasted conditions on Days 10 and 13. The up-titration regimen consists of 2 qd doses of 10 mg macitentan and 3 qd doses of 37.5 mg macitentan followed by 8 qd doses of 75 mg macitentan. On Days −4, −3, 10 and 13 (i.e., the days when dosing occurred under fasted conditions), no fluid intake apart from the water taken at the time of macitentan administration was allowed from 1 hour before until 1 hour after administration of macitentan.

Subjects received standardized meals and on dosing days when macitentan was administered in fed state, at the following time points:
Evening meal not later than 10 hours before macitentan administration next day.
Breakfast within 30 minutes prior to macitentan administration (Days 1-9 and Days 11-12).
Lunch approximately 4 hours after macitentan administration.
Snack approximately 7 hours after macitentan administration.
Evening meal approximately 10 hours after macitentan administration.

C. Safety Measurements

Tables 1A, 1 Ba and 1Bb summarize the frequency and timing of safety measurements.

TABLE 1A
Screening Phase

| Study Day | Days −25 to −6 |
|---|---|
| Physical examination | X |
| Weight/Height (kg/cm) | X |
| Electrocardiogram (ECG) ^{b} | X |
| Vital signs ^{b} | X |
| Serology | X |
| eGFR^{f} | X |
| Hematology, chemistry, and urinalysis ^{d} | X |

TABLE 1Ba
Treatment Phase (without up-titration)

| | Study Day | | |
|---|---|---|---|
| | Day 1 | | |
| Time | Predose | 0 | Day 2-9 |
| Physical examination | X | | Day 9 |
| Symptom-directed physical | X | | Days 2-9 |
| examination | | | |
| Weight (kg) | X | | Days 2-9 |
| ECG ^{a} | X | | Day 9 |
| Vital signs ^{a,b} | X | | Days 3, 5, 7, 9 |
| Hematology, chemistry, | X | | Days 5, 9 ^{c} |
| and urinalysis ^{d} | | | |
| Macitentan dosing ^{e} | | X ^{c} | Days 2-9 |

| | |
|---|---|
| Adverse events | continuous |

^{a} Vital signs and ECGs measured after 5 minutes rest in supine position, the ECG using a standard 12-lead ECG. Blood pressure measured after approximately 2 minutes in standing position.
^{b} Vital signs include blood pressure (supine and standing), pulse rate, respiratory rate and temperature. Blood pressure and respiratory rate measured after 5 minutes rest in supine position. Standing blood pressure measured after approximately 2 minutes in standing position. Respiratory rate measured over at least 30 seconds.
^{c} Subjects fasted for at least 10 hours before dosing. Water (except for the 240 mL used for dosing) was restricted from 1 hour prior to until 1 hour after macitentan administration.
^{d} Subjects fasted for at least 10 hours.
^{e} Dosing under fed conditions.
^{f} eGFR was calculated according to the MDRD equation.

TABLE 1Bb
Treatment Phase (with up-titration)

| | Study Day | | | | |
|---|---|---|---|---|---|
| | Day 1 | | | | |
| Time | Predose | 0 | Day 2-9 | Day 10-12 | Day 13 |
| Physical | X | | Day 9 | X | X |
| examination | | | | | |
| Weight | X | | Days 2-9 | X | X |
| ECG^{(a),(b)} | X | | Day 9 | X | X |
| Vital signs^{(a),(b)} | X | | Days | X | X |
| | | | 3, 5, 7, 9 | | |
| Drug/alcohol screen | X | | | | |
| Hematology, | X | | Days 5 | X | X |
| chemistry, and | | | and 9^{(e)} | | |
| urinalysis^{(a),(d)} | | | | | |
| Macitentan dosing | | X | X | X | X |

| | |
|---|---|
| Adverse events | continuous |

^{(a)}Vital signs and ECGs measured after 5 minutes rest in supine position, the ECG using a standard 12-lead ECG. Blood pressure measured after approximately 2 minutes in standing position.
^{(b)}Vital signs include blood pressure (supine and standing), pulse rate, respiratory rate and temperature. Blood pressure and respiratory rate measured after 5 minutes rest in supine position. Standing blood pressure measured after approximately 2 minutes in standing position. Respiratory rate measured over at least 30 seconds.
^{(c)}Subjects fasted for at least 10 hours before dosing. Water (except for the 240 mL used for dosing) was restricted from 1 hour prior to until 1 hour after macitentan administration.
^{(d)}Subjects fasted for at least 10 hours.
^{(e)}Dosing under fed conditions.
^{(f)}eGFR was calculated according to the MDRD equation.

The following tests were performed (Table 2):

TABLE 2
Hematology Panel

| | | |
|---|---|---|
| haemoglobin | platelet count | RBC count |
| haematocrit | differential blood count | WBC count with |
| | | differential |

Serum Chemistry Panel

| | | |
|---|---|---|
| sodium | Creatinine | lactic acid |
| | | dehydrogenase |
| potassium | Glucose | creatine phosphokinase |
| chloride | AST | uric acid |
| calcium | ALT | albumin |
| bicarbonate | gamma- | total protein |
| | glutamyltransferase | |
| phosphate | total, indirect and direct | total cholesterol |
| | bilirubin | |
| urea | alkaline phosphatase | triglycerides |

Urinalysis

| | | |
|---|---|---|
| Dipstick | Ketones | epithelial cells |
| specific gravity | Bilirubin | crystals |
| pH | Urobilinogen | casts |
| glucose | Sediment | bacteria |
| protein | RBC | nitrite |
| blood | WBC | leukocyte esterase |

D. Safety Analyses

All reported AEs with onset during the treatment phase were included in the analysis. The effects on cardiovascular variables were evaluated and the following variables were collected: PR (ms), QRS (ms), QT (ms), QTcB (ms), QTcF (ms), heart rate (bpm), and RR. The normal range for vital signs is defined as follows:
Systolic blood pressure: 100-140 mmHg
Diastolic blood pressure: 60-90 mmHg
Pulse rate: 45-90 bpm
Respiratory rate: 8-20 per minute
Body temperature: 35.0° C. to 37.5° C.

An AE is any untoward medical occurrence, i.e., any unfavorable and unintended sign (including an abnormal finding), symptom, or disease temporally associated with the use of macitentan. A treatment-emergent AE is any occurrence that is new in onset or aggravated in severity from the baseline condition. The following are used to assess all AEs:
Related: There is a reasonable causal relationship between macitentan administration and the AE.
Not Related: There is not a reasonable causal relationship between macitentan administration and the AE.

An assessment of AE severity grade was made using the following general categorical descriptors:
Mild: Awareness of symptoms that are easily tolerated, causing minimal discomfort and not interfering with everyday activities.
Moderate: Sufficient discomfort is present to cause interference with normal activity.
Severe: Extreme distress, causing significant impairment of functioning or incapacitation. Prevents normal everyday activities.

E. Results

The AEs for subjects in the first cohort with and without up-titration were then analyzed. See, Table 4.

TABLE 4

| | | | | | Frequency |
|---|---|---|---|---|---|
| | | Severity of | | Severity of | observed with |
| | | AEs | | AEs | macitentan |
| | N | (No titration) | N | (With titration) | 10 mg |
| Total number of | 6 | | 6 | | |
| patients | | | | | |
| Headache | 6 | 5 mild | 6 | 5 mild | Very common |
| | | 1 moderate | | 1 moderate | |
| Sinusitis/rhinitis/ | 5 | All mild | 1 | Mild, less than | Common |
| nasal congestion | | | | 24 hours | |
| Flushing/feeling | 4 | 3 mild | 0 | | Uncommon |
| of warmth/ | | 1 moderate | | | |
| redness of face | | | | | |
| and/or hands | | | | | |
| Photophobia | 3 | All mild | 0 | | Not known |

Frequencies are defined as: very common (≥1/10); common (≥1/100 to <1/10); uncommon (≥1/1,000 to <1/100); rare (≥1/10,000 to <1/1,000); very rare (<1/10,000); not known (cannot be estimated from the available data).

AEs were reported from Day 2 onwards, and were mainly mild. Four days after stopping macitentan most of the AEs resolved. For vital signs, ECG and lab parameters, no clinically significant changes reported. Further, a reduction in hemoglobin observed, within the expected range (No decrease >2 g/dL).

In summary, the data show that the introduction of an up-titration regimen improved tolerability of 75 mg macitentan. All subjects completed treatment with macitentan in the up-titration regimen cohort.

1-28. (canceled)

29. A method for treating pulmonary vascular disease and/or cardiac dysfunction in a Fontan-palliated patient, comprising administering to the patient in need thereof macitentan at a dosage of 60 mg to 90 mg per day.

30. The method of claim 29, wherein the dosage is 70 mg to 80 mg per day.

31. The method of claim 30, wherein the dosage is 35 mg to 40 mg twice per day.

32. The method of claim 29, wherein the dosage is 75 mg per day.

33. The method of claim 32, wherein the dosage is 37.5 mg twice per day.

34. The method of claim 29, wherein the method reduces a morbidity risk, a mortality risk, or both, of the pulmonary vascular disease and/or cardiac dysfunction.

35. The method of claim 29, further comprising administering to the patient a PDE5 inhibitor, a prostacyclin analogue, a prostacyclin receptor agonist, or a soluble guanylate cyclase stimulator, or a combination thereof.

36. The method of claim 35, wherein the PDE5 inhibitor is sildenafil, tadalafil, vardenafil, or udenafil; the prostacyclin analogue is epoprostenol, treprostinil, iloprost, or beraprost; the prostacyclin receptor agonist is selexipag or ralinepag; and the soluble guanylate cyclase stimulator is riociguat or vericiguat.

37. The method of claim 29, further comprising administering to the patient tadalafil, selexipag, or ralinepag, or a combination thereof.

38. The method of claim 37, wherein the tadalafil is administered at a dosage of 20 mg to 40 mg per day, the selexipag is administered at a dosage of 0.2 mg to 1.6 mg twice per day, or the ralinepag is administered at a dosage of 0.05 mg to 1.45 mg per day, or a combination thereof.

39. The method of claim 38, wherein the dosage of tadalafil is 40 mg per day.

40. The method of claim 29, further comprising administering to the patient tadalafil or selexipag, or a combination thereof.

41. The method of claim 29, wherein the patient is already being treated with an endothelin receptor antagonist prior to administering macitentan.

42. The method of claim 41, wherein the endothelin receptor antagonist is bosentan or ambrisentan.

43. The method of claim 29 wherein the macitentan is administered in a pharmaceutical composition, the pharmaceutical composition comprising:
10% to 50% by weight of macitentan, based on the total weight of the pharmaceutical composition;
10% to 85% by weight of a filler, based on the total weight of the pharmaceutical composition;
1% to 10% by weight of a disintegrant, based on the total weight of the pharmaceutical composition;
0.1% to 1% by weight of a surfactant, based on the total weight of the pharmaceutical composition; and
0.05% to 5% by weight of a lubricant, based on the total weight of the pharmaceutical composition.

44. The method of claim 24, wherein the filler comprises lactose monohydrate and microcrystalline cellulose; the disintegrant comprises sodium starch glycolate or a combination of sodium starch glycolate and polyvinylpyrrolidone; the surfactant comprises a polysorbate; and the lubricant comprises magnesium stearate.

45. The method of claim 24, wherein the pharmaceutical composition is in the form of a capsule or tablet.
